(19) United States
(12) Patent Application Publication
(10) Pub. No.: US 2010/0117066 A1
(43) Pub. Date: May 13, 2010

(54) Organic Semiconductors

(75) Inventors: Martin Heeney, Southampton (GB); Weinmin Zhang, Southampton (GB); Iain McCullooch, Southampton (GB)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstradt (DE)

(21) Appl. No.: 12/597,832

(22) PCT Filed: Mar. 28, 2008
(86) PCT No.: PCT/EP2008/002483

§ 371 Date: Oct. 27, 2009

(30) Foreign Application Priority Data
Apr. 28, 2007 (EP) 07008725.9

(51) Int. Cl. H01L 51/30; C07D 495/04

(52) U.S. Cl. 257/40; 549/41; 549/4; 546/256; 548/524; 257/E51.027

(57) ABSTRACT

The invention relates to novel substituted dibenzo[d,d′]benzo[1,2-b;4,5-b′]dithiophenes (DBBDT), to methods of their synthesis, to organic semiconducting materials, formulations and layers comprising them, and to electronic devices, like organic field effect transistors (OFETs), comprising them.

### FIELD OF THE INVENTION

The invention relates to novel substituted dibenzo[d,d′]benzo[1,2-b;4,5-b′]dithiophenes (DBBDT), to methods of their synthesis, to organic semiconducting materials, formulations and layers comprising them, and to electronic devices, like organic field effect transistors (OFETs), comprising them.

### BACKGROUND AND PRIOR ART

6,13-Diethynylsubstituted pentacene derivatives have found use as solution-processable organic semiconductors. For example, 6,13-bis(triisopropylsilylethynyl)pentacene 1,

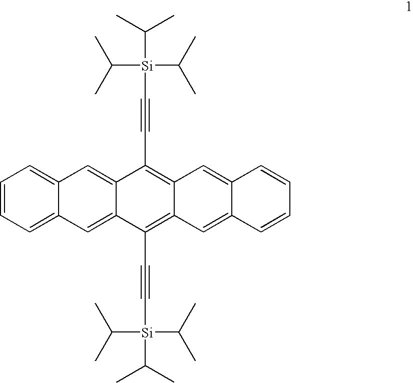

has been reported to exhibit high solubility (>100 mg/mL in chloroform) and yield organic field-effect transistor (OFET) devices with hole mobility of 0.17 cm²/V·s and an on/off current ratio of 10⁵ when fabricated from solution (see J. Am. Chem. Soc, 2005, 127, 4986). However, substituted pentacenes exhibit poor photostability, both in solution and in the solid state, undergoing [4+4] dimerisations and photooxidations. (see Adv. Mater. 2005, 3001). In addition, pentacene 1 exhibits an undesirable thermal transition at 124° C. This is related to a crystal to crystal phase transition. In thin films of 1, this can lead to thermal expansion and cracking, if the film is heated above this transition. (see J. Chem. Phys. B. 2006, 110, 16397) In a field effect device, such cracking causes a substantial decrease in performance. The temperature this phase transition occurs is undesirably low for device manufacture so structures with increased thermal stability are desirable.

It was therefore an aim of the present invention to provide compounds for use as organic semiconducting materials that do not have the drawbacks of prior art materials as described above, and do especially show good processability, good solubility in organic solvents and high charge carrier mobility. Another aim of the invention was to extend the pool of organic semiconducting materials available to the expert. Other aims of the present invention are immediately evident to the expert from the following detailed description.

It was found that these aims can be achieved by providing compounds as claimed in the present invention. The inventors of the present invention have found that substituted dibenzo[d,d′]benzo[1,2-b;4,5-b′]dithiophenes (DBBDT, 2)

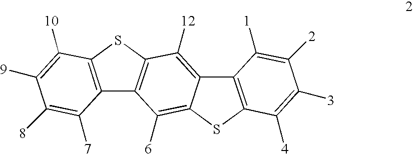

can be used as semiconductors that exhibit very good solubility in most organic solvents, and show high performance when used as semiconducting layer in an organic field-effect transistor (OFET), with charge carrier mobility of 0.1-0.5 cm²/V·s and current on/off ratio of 10⁵ by solution deposition fabrication.

### SUMMARY OF THE INVENTION

The invention relates to compounds of formula I

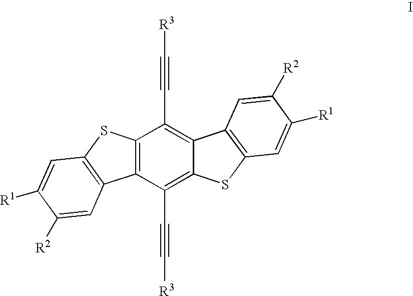

wherein
R¹, R² and R³ are independently of each other halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, optionally substituted silyl groups, or optionally substituted carbyl or hydrocarbyl groups that optionally comprise one or more hetero atoms, neighboured groups R¹ and R² may also form a ring system with each other or with the benzene ring to which they are attached, and R¹ and/or R² may also denote H,
X⁰ is halogen,
R⁰ and R⁰⁰ are independently of each other H or an optionally substituted aliphatic or aromatic hydrocarbyl group having 1 to 20 C atoms.

The invention further relates to a semiconductor or charge transport material, component or device comprising one or more compounds of formula I.

The invention further relates to a formulation comprising one or more compounds of formula I and one or more solvents, preferably selected from organic solvents.

The invention further relates to an organic semiconducting formulation comprising one or more compounds of formula I, one or more organic binders, or precursors thereof, preferably having a permittivity ∈ at 1,000 Hz of 3.3 or less, and optionally one or more solvents.

The invention further relates to the use of compounds and formulations according to the present invention as charge transport, semiconducting, electrically conducting, photoconducting or light emitting material in an optical, electrooptical, electronic, electroluminescent or photoluminescent components or devices.

The invention further relates to a charge transport, semiconducting, electrically conducting, photoconducting or light emitting material or component comprising one or more compounds or formulations according to the present invention.

The invention further relates to an optical, electrooptical, electronic, electroluminescent or photoluminescent component or device comprising one or more compounds or formulations according to the present invention.

Said components and devices include, without limitation, electrooptical displays, LCDs, optical films, retarders, compensators, polarisers, beam splitters, reflective films, alignment layers, colour filters, holographic elements, hot stamping foils, coloured images, decorative or security markings, LC pigments, adhesives, non-linear optic (NLO) devices, optical information storage devices, electronic devices, organic semiconductors, organic field effect transistors (OFET), integrated circuits (IC), thin film transistors (TFT), Radio Frequency Identification (RFID) tags, organic light emitting diodes (OLED), organic light emitting transistors (OLET), electroluminescent displays, organic photovoltaic (OPV) devices, organic solar cells (O-SC), organic laser diodes (O-laser), organic integrated circuits (O-IC), lighting devices, sensor devices, electrode materials, photoconductors, photodetectors, electrophotographic recording devices, capacitors, charge injection layers, Schottky diodes, planarising layers, antistatic films, conducting substrates, conducting patterns, photoconductors, electrophotographic or electrophotographic recording devices, organic memory devices, biosensors and biochips.

### DETAILED DESCRIPTION OF THE INVENTION

The inclusion of five membered heteroaromatics such as thiophene into the linear backbone of pentacene results in a slight non-linearity of the backbone. It is known from prior art that acenes having all of their benzene rings fused in a linear array are unstable compared with their non-linear structural isomers that have the same number of benzene rings [see (a) Clar, E. The Aromatic Sextet; Wiley-Interscience: London, 1972. (b) Suresh, C. H.; Gadre, S. R. J. Org. Chem. 1999, 64, 2505-2512. (c) Aihara, J. J. Am. Chem. Soc. 2006, 128, 2873-2879]. In contrast, the DBBDT's according to the present invention show enhanced photo and thermal stability, both in solution and in the solid state.

The introduction of bulky substituents such as trialkylsilylethynyl groups at the C-6/C-12 positions of dibenzo[d,d′]benzo[1,2-b;4,5-b′]dithiophenes has proven to be effective for solubilization. Arylethynyl substituents can also be introduced, to both promote solubility and enhance pi-electron delocalization from the core. Substituents can also be introduced at the 2,8 or 3,9 positions to further tune the molecular properties.

The term “carbyl group” as used above and below denotes any monovalent or multivalent organic radical moiety which comprises at least one carbon atom either without any non-carbon atoms (like for example —C≡C—), or optionally combined with at least one non-carbon atom such as N, O, S, P, Si, Se, As, Te or Ge (for example carbonyl etc.). The term “hydrocarbyl group” denotes a carbyl group that does additionally contain one or more H atoms and optionally contains one or more hetero atoms like for example N, O, S, P, Si, Se, As, Te or Ge.

A carbyl or hydrocarbyl group comprising a chain of 3 or more C atoms may also be straight-chain, branched and/or cyclic, including spiro and/or fused rings.

Preferred carbyl and hydrocarbyl groups include alkyl, alkoxy, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from N, O, S, P, Si, Se, As, Te and Ge.

The carbyl or hydrocarbyl group may be a saturated or unsaturated acyclic group, or a saturated or unsaturated cyclic group. Unsaturated acyclic or cyclic groups are preferred, especially aryl, alkenyl and alkynyl groups (especially ethynyl). Where the C₁-C₄₀ carbyl or hydrocarbyl group is acyclic, the group may be straight-chain or branched. The C₁-C₄₀ carbyl or hydrocarbyl group includes for example: a C₁-C₄₀ alkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₂-C₂₀ alkenyl group, a C₂-C₂₀ alkynyl group, a C₃-C₂₀ allyl group, a C₄-C₂₀ alkyldienyl group, a C₆-C₁₂ aryl group and a C₄-C₂₀ polyenyl group, respectively. Also included are combinations of groups having carbon atoms and groups having hetero atoms, like e.g. an alkynyl group, preferably ethynyl, that is substituted with a silyl group, preferably a trialkylsilyl group.

Aryl and heteroaryl preferably denote a mono-, bi- or tricyclic aromatic or heteroaromatic group with up to 25 C atoms that may also comprise condensed rings and is optionally substituted with one or more groups L. Preferred substituents L are selected from F, Cl, Br, I, —CN, —NO₂, —NCO, —NCS, —OCN, —SCN, —C(═O)NR⁰R⁰⁰, —C(═O)X⁰, —C(═O)R⁰, —NR⁰R⁰⁰, optionally substituted silyl, or aryl or heteroaryl with 4 to 40, preferably 6 to 20 ring atoms, and straight chain or branched alkyl, alkoxy, oxaalkyl, thioalkyl, alkenyl, alkynyl, alkylcarbonyl, alkoxycarbonyl, alkylcarbonlyoxy or alkoxycarbonyloxy with 1 to 20, preferably 1 to 12 C atoms, wherein one or more H atoms are optionally replaced by F or Cl, wherein R⁰ and R⁰⁰ are as defined above and X⁰ is halogen.

Very preferred substituents L are selected from halogen, most preferably F, or alkyl, alkoxy, oxaalkyl, thioalkyl, fluoroalkyl and fluoroalkoxy with 1 to 12 C atoms or alkenyl, alkynyl with 2 to 12 C atoms.

Especially preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, pyridine, preferably 2- or 3-pyridine, pyrimidine, thiophene preferably 2-thiophene, selenophene, preferably 2-selenophene, thieno[3,2-b]thiophene, thiazole, thiadiazole, oxazole and oxadiazole, especially preferably thiophene-2-yl, 5-substituted thiophene-2-yl or pyridine-3-yl, all of which can be unsubstituted, mono- or polysubstituted with L as defined above.

Especially preferred are compounds of formula I wherein one or more of R¹ and R² denote aryl or heteroaryl optionally substituted by L as defined above, or straight chain, branched or cyclic alkyl with 1 to 20 C-atoms, which is unsubstituted or mono- or polysubstituted by F, Cl, Br or I, and wherein one or more non-adjacent CH₂ groups are optionally replaced, in each case independently from one another, by —O—, —S—, —NR⁰—, —SiR⁰R⁰⁰—, —CY¹═CY²— or —C≡C— in such a manner that O and/or S atoms are not linked directly to one another, or denotes optionally substituted aryl or heteroaryl preferably having 1 to 30 C-atoms, with
R⁰ and R⁰⁰ being independently of each other H or alkyl with 1 to 12 C-atoms,
Y¹ and Y² being independently of each other H, F, Cl or CN,

Further preferred are compounds of formula I wherein one or more groups R¹ and R², preferably both groups R¹, are selected of formula -(A-B)ₐ, wherein, in case of multiple occurrence independently of one another, A is selected from —CY¹═CY²— or —C≡C— and B is selected from aryl or heteroaryl optionally substituted by L as defined above, with Y¹ and Y² being as defined above, and a being 1, 2 or 3.

Further preferred are compounds of formula I wherein one or more groups R¹ and R² denote C₁-C₂₀-alkyl that is optionally substituted with one or more fluorine atoms, C₁-C₂₀-alkenyl, C₁-C₂₀-alkynyl, C₁-C₂₀-alkoxy or -oxaalkyl, C₁-C₂₀-thioalkyl, C₁-C₂₀-silyl, C₁-C₂₀-amino or C₁-C₂₀-fluoroalkyl, in particular from alkenyl, alkynyl, alkoxy, thioalkyl or fluoroalkyl, all of which are straight-chain and have 1 to 12, preferably 5 to 12 C-atoms, most preferably pentyl, hexyl, heptyl, octyl, nonyl, decyl, undecyl or dodecyl.

If two or more of the substituents R^{1,2} form a ring system with each other or with the benzene ring to which they are attached, this is preferably a 5-, 6- or 7-membered aromatic or heteroaromatic ring, preferably selected from pyrrole, pyridine, pyrimidine, thiophene, selenophene, thiazole, thiadiazole, oxazole and oxadiazole, especially preferably thiophene or pyridine, all of which are optionally substituted by L as defined above.

Especially preferred are compounds of formula I, wherein one or both groups R³ denote a silyl group, or an optionally substituted aryl or heteroaryl group, preferably optionally substituted by L as defined above.

The silyl group is optionally substituted and is preferably selected of the formula —SiR′R″R′″. Therein, R′, R″ and R′″ are identical or different groups selected from H, a C₁-C₄₀-alkyl group, preferably C₁-C₄-alkyl, most preferably methyl, ethyl, n-propyl or isopropyl, a C₂-C₄₀-alkenyl group, preferably C₂-C₇-alkenyl, a C₆-C₄₀-aryl group, preferably phenyl, a C₆-C₄₀-arylalkyl group, a C₁-C₄₀-alkoxy or -oxaalkyl group, or a C₆-C₄₀-arylalkyloxy group, wherein all these groups are optionally substituted with one or more groups L as defined above. Preferably, R′, R″ and R′″ are each independently selected from optionally substituted C₁₋₁₀-alkyl, more preferably C₁₋₄-alkyl, most preferably C₁₋₃-alkyl, for example isopropyl, and optionally substituted C₆₋₁₀-aryl, preferably phenyl. Further preferred is a silyl group wherein one or more of R′, R″ and R′″ form a cyclic silyl alkyl group together with the Si atom, preferably having 1 to 8 C atoms.

In one preferred embodiment of the silyl group, R′, R″ and R′″ are identical groups, for example identical, optionally substituted, alkyl groups, as in triisopropylsilyl. Very preferably the groups R′, R″ and R′″ are identical, optionally substituted C₁₋₁₀, more preferably C₁₋₄, most preferably C₁₋₃ alkyl groups. A preferred alkyl group in this case is isopropyl.

A silyl group of formula —SiR′R″R′″ or —SiR′R″″ as described above is a preferred optional substituent for the C₁-C₄₀-carbyl or hydrocarbyl group.

Preferred groups —SiR′R″R′″ include, without limitation, trimethylsilyl, triethylsilyl, tripropylsilyl, dimethylethylsilyl, diethylmethylsilyl, dimethylpropylsilyl, dimethylisopropylsilyl, dipropylmethylsilyl, diisopropylmethylsilyl, dipropylethylsilyl, diisopropylethylsilyl, diethylisopropylsilyl, triisopropylsilyl, trimethoxysilyl, triethoxysilyl, trimethoxymethylsilyl, trivinylsilyl, triphenylsilyl, diphenylisopropylsilyl, diisopropylphenylsilyl, diphenylethylsilyl, diethylphenylsilyl, diphenylmethylsilyl, triphenoxysilyl, dimethylmethoxysilyl, dimethylphenoxysilyl, methylmethoxyphenylsilyl, etc., wherein the alkyl, aryl or alkoxy group is optionally substituted.

An alkyl or alkoxy radical, i.e. where the terminal CH₂ group is replaced by —O—, can be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, nonoxy, decoxy, undecoxy, dodecoxy, tridecoxy or tetradecoxy, for example.

An alkenyl group, wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example. Oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one by —CO—, these radicals are preferably neighboured. Accordingly these radicals together form a carbonyloxy group —CO—O— or an oxycarbonyl group —O—CO—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxy-ethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxy-carbonylmethyl, propoxycarbonylmethyl, butoxycarbonylmethyl, 2-(methoxycarbonyl)ethyl, 2-(ethoxycarbonyl)ethyl, 2-(propoxy-carbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —COO— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e where one CH₂ group is replaced by —S—, is preferably straight-chain thiomethyl (—SCH₃), 1-thioethyl (—SCH₂CH₃), 1-thiopropyl (=—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is preferably straight-chain perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₅F₁₇, very preferably C₆F₁₃.

R¹⁻³ and R′, R″, R′″ can be an achiral or a chiral group. Particularly preferred chiral groups are 2-butyl (=1-methylpropyl), 2-methylbutyl, 2-methylpentyl, 3-methylpentyl, 2-ethylhexyl, 2-propylpentyl, in particular 2-methylbutyl, 2-methylbutoxy, 2-methylpentoxy, 3-methylpentoxy, 2-ethylhexoxy, 1-methylhexoxy, 2-octyloxy, 2-oxa-3-methylbutyl, 3-oxa-4-methylpentyl, 4-methylhexyl, 2-hexyl, 2-octyl, 2-nonyl, 2-decyl, 2-dodecyl, 6-methoxyoctoxy, 6-methyloctoxy, 6-methyloctanoyloxy, 5-methylheptyloxycarbonyl, 2-methylbutyryloxy, 3-methylvaleroyloxy, 4-methylhexanoyloxy, 2-chlorpropionyloxy, 2-chloro-3-methylbutyryloxy, 2-chloro-4-methylvaleryloxy, 2-chloro-3-methylvaleryloxy, 2-methyl-3-oxapentyl, 2-methyl-3-oxahexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

—CY¹═CY²— is preferably —CH═CH—, —CF═CF— or —CH═C(CN)—.

Halogen is F, Cl, Br or I, preferably F, Cl or Br.

Especially preferred are the compounds of the following subformulae:

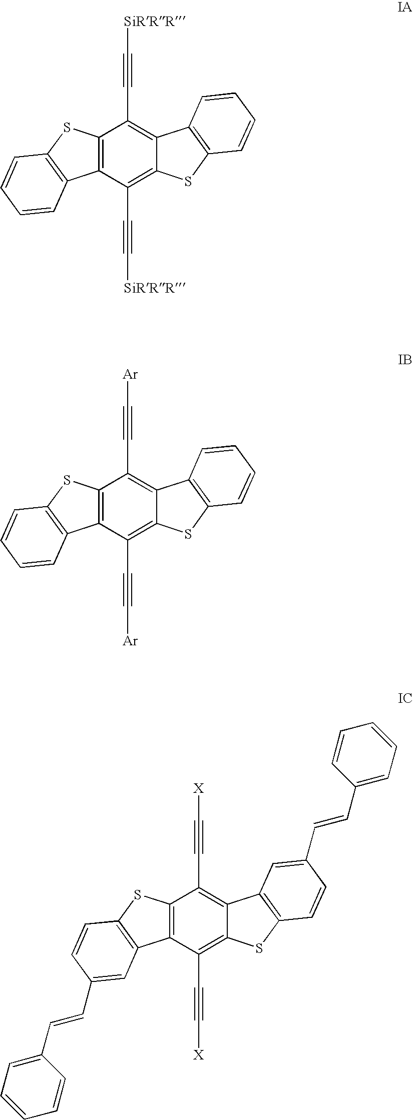

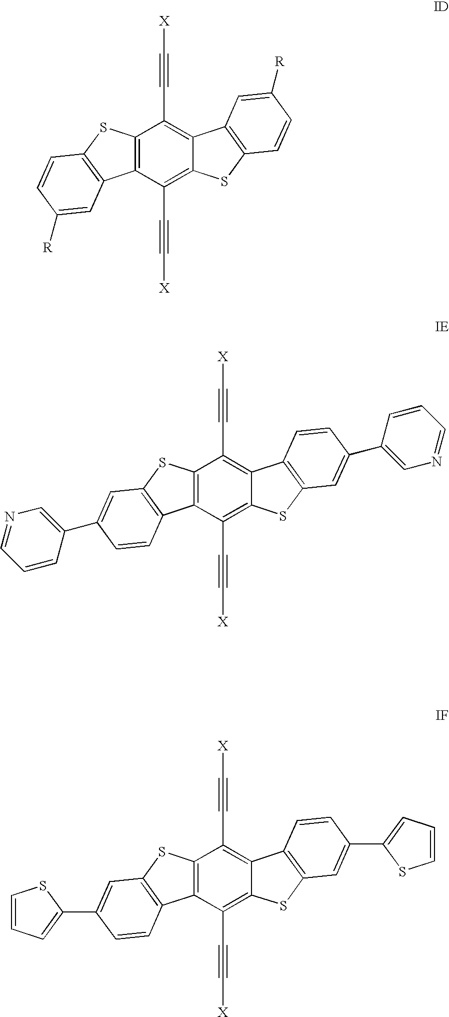

wherein R′, R″ and R′″ are as defined above, R has one of the meanings of R² given above different from H, X denotes SiR′R″R′″ or Ar, and Ar is in each occurrence independently of one another aryl or heteroaryl group optionally substituted by L as defined above.

Further preferred are compounds of the following subformulae:

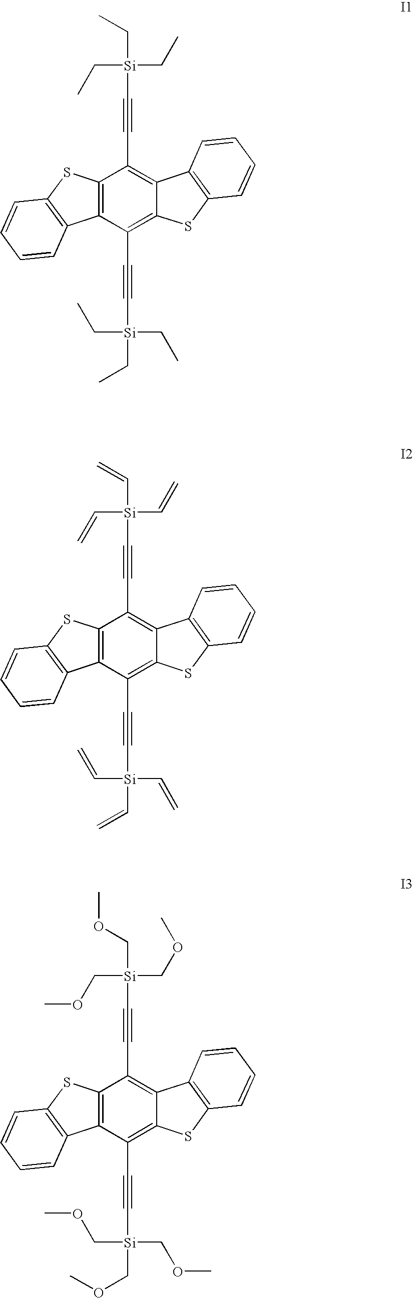

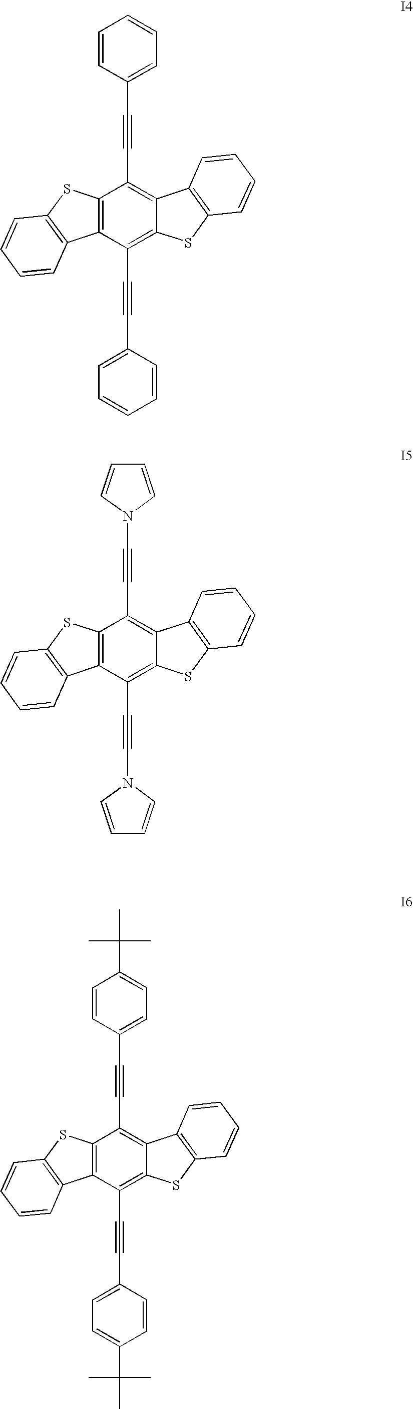

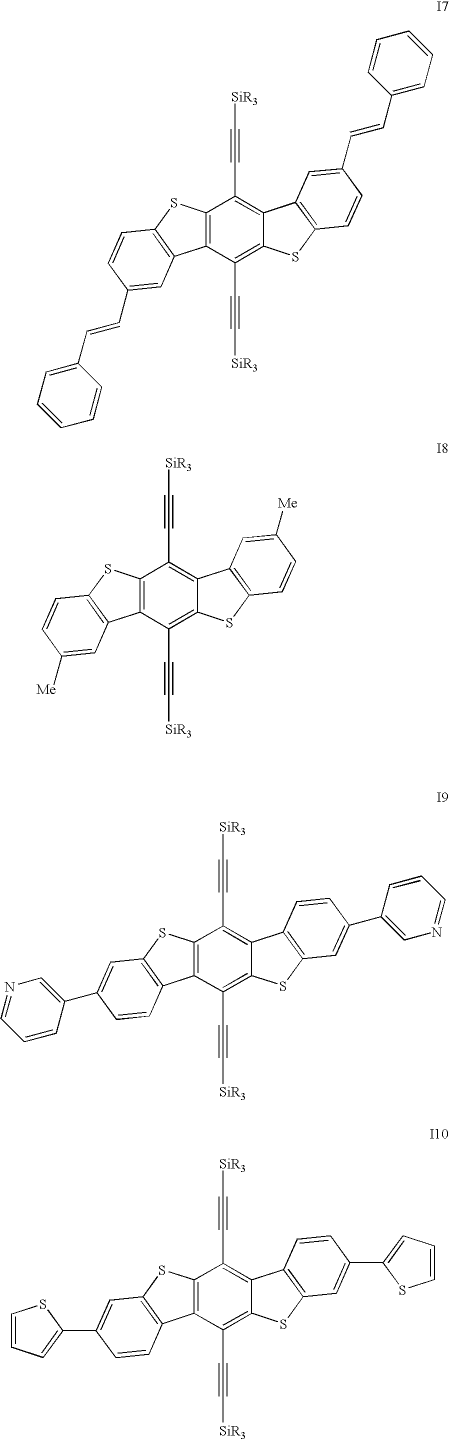

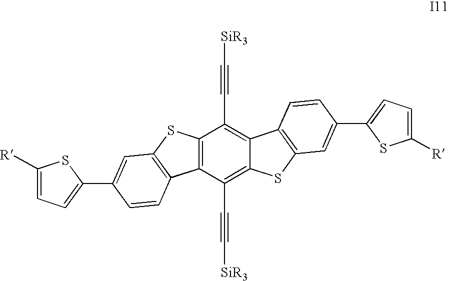

wherein R has one of the meanings of R′ given above.

The compounds of the present invention can be synthesized according to or in analogy to known methods or to the methods described below. Further methods can be taken from the examples.

Especially suitable and preferred methods for preparing substituted compounds of formula I are shown in the following reaction schemes.

The methods of preparing a compound of formula I are another aspect of the invention. Especially preferred is a method comprising the following steps:

a) treatment of an optionally substituted benzo[b]thiophene-3-carboxylic acid dialkylamide or naphtho[2,3-b]thiophene-3-carboxylic acid dialkylamide with at least one equivalent of a strong base at low temperature. The base should be of sufficient strength to deprotonate the 2-position of the benzo[b]thiophene or naphtho[2,3-b]thiophene. Examples include n-butyllithium (BuLi), sec-butyllithium, tert-butyllithium lithium diisopropylamide (LDA), lithium tetramethylpiperidide (LiTMP) or lithium hexamethyldisilazane (LiHMDS). Subsequent heating of this organolithium intermediate promotes an intramolecular condensation reaction between the aryllithium and the carboxylic dialkylamide of another molecule to generate a substituted ketone. A second intermolecular reaction between the aryllithium and carboxylic acid dialkylamide of the resulting ketone affords a fused aromatic quinone (an optionally substituted dibenzo[d,d′]benzo[1,2-b;4,5-b′]dithiophene-6,12-dione),
b) treatment of the resultant fused aromatic quinone with at least two equivalents of an optionally substituted alkynyl lithium or alkynyl magnesium reagent. Nucleophillic addition of the organometallic species to the carbonyl groups results in the generation of a diol intermediate. Acidic work-up of the resulting diol, optionally in the presence of a reducing agent such as tin (II) chloride or sodium iodide/sodium hypophosphite, results in generation of the desired molecules of formula I.

The synthesis of unsubstituted dibenzo[d,d′]benzo[1,2-b;4,5-b′]dithiophenes (DBBDT) is outlined in Scheme 1. Benzo[b]thiophene-3-carboxylic acid 4 is converted to bromobenzo[b]thiophene-3-carboxylic acid dimethylamide 5 by any one of variety of known amide formation reactions. Subsequent treatment of this amide with an alkyl lithium reagent at low temperature deprotonates the 2-position, generating the organolithium reagent. Heating of this intermediate promotes an intramolecular condensation reaction with another carboxylic acid dimethylamide to generate a diaryl ketone. A second intermolecular reaction between the aryllithium and the carboxylic acid dimethylamide of the resulting ketone generates the quinone (dibenzo[d,d′]benzo[1,2-b;4,5-b′]dithiophene-6,12-dione 6). This process is in analogy to that reported by Slocum and Gierer (see J. Org. Chem. 1976, 3668). The synthesis of 6 by this route has not been reported previously, although it has been reported by other routes (see J. Heter. Chem. 1997, 34, 781-787). Finally introduction of the ethynyl functional group can be achieved by reacting 6 with an excess of lithium or magnesium acetylide, followed by dehydration with tin (II) chloride in an analogous manner to that described by Anthony and co-workers (see J. Am. Chem. Soc, 2005, 127, 4986), to give target molecule 7.

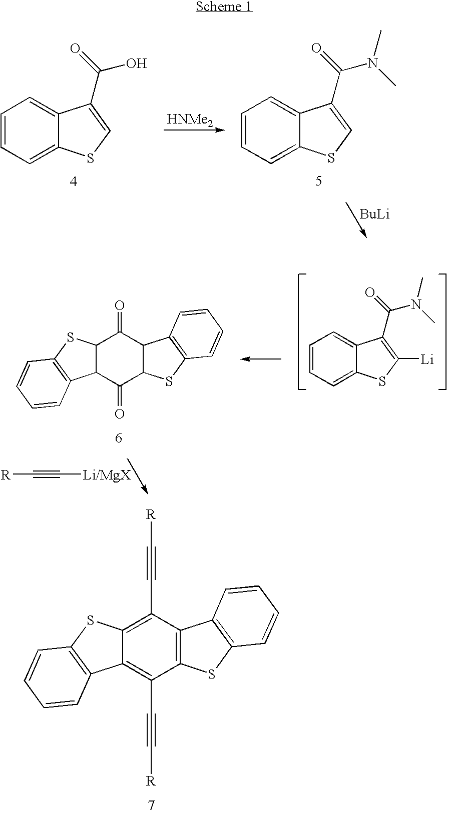

Substituents maybe introduced onto the periphery of the DBBDT core in the 3,9 or 2,8 positions by the use of 6-bromobenzothiophene[b]carboxylic acid (see J. Med. Chem. 2003, 46, 2446-2455.) or 5-bromobenzothiophene[b]carboxylic acid (commercially available) as starting materials (see Scheme 2). Thus conversion of the brominated carboxylic acid to the dimethylamide, can be followed by the transition metal catalysed reaction of the aryl bromide to introduce a variety of aryl, alkyl, alkenyl or alkynyl substituents, optionally substituted. Subsequent reaction with an organolithium intermediate generates the quinone, which can be further reacted as described above.

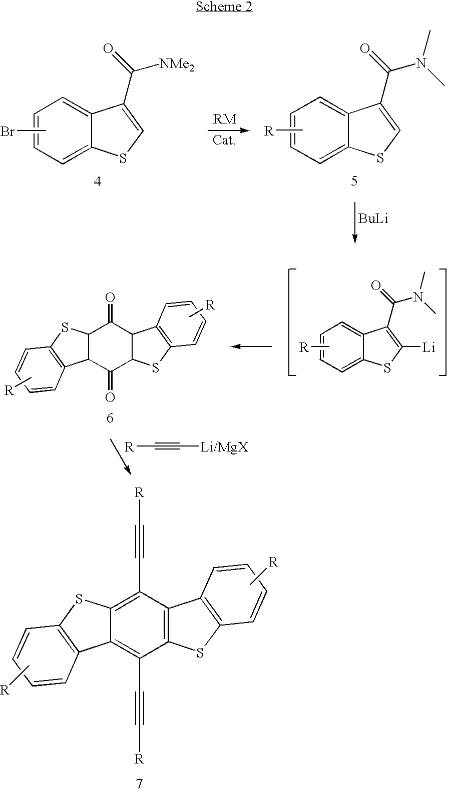

The invention further relates to a formulation comprising one or more compounds of formula I and one or more solvents, preferably selected from organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetramethyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, dimethylformamide, 2-chloro-6fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylansiole, 3-methylanisole, 4-fluoro-3-methylanisole, 2-fluorobenzonitrile, 4-fluoroveratrol, 2,6-dimethylanisole, 3-fluorobenzonitrile, 2,5-dimethylanisole, 2,4-dimethylanisole, benzonitrile, 3,5-dimethylanisole, N,N-dimethylaniline, ethyl benzoate, 1-fluoro-3,5-dimethoxybenzene, 1-methylnaphthalene, N-methylpyrrolidinone, 3-fluorobenzotrifluoride, benzotrifluoride, benzotrifluoride, diosane, trifluoromethoxybenzene, 4-fluorobenzotrifluoride, 3-fluoropyridine, toluene, 2-fluorotoluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluorobenzene, 3-chlorofluorobenzene, 1-chloro-2,5-difluorobenzene, 4-chlorofluorobenzene, chlorobenzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents with high boiling temperatures and solvent mixtures are preferred. For spin coating alkylated benzenes like xylene and toluene are preferred.

The invention further relates to an organic semiconducting formulation comprising one or more compounds of formula I, one or more organic binders, or precursors thereof, preferably having a permittivity ∈ at 1,000 Hz of 3.3 or less, and optionally one or more solvents.

Combining specified soluble compounds of formula I, especially compounds of the preferred formulae as described above and below, with an organic binder resin (hereinafter also referred to as “the binder”) results in little or no reduction in charge mobility of the compounds of formula I, even an increase in some instances. For instance, the compounds of formula I may be dissolved in a binder resin (for example poly(α-methylstyrene) and deposited (for example by spin coating), to form an organic semiconducting layer yielding a high charge mobility. Moreover, a semiconducting layer formed thereby exhibits excellent film forming characteristics and is particularly stable.

If an organic semiconducting layer formulation of high mobility is obtained by combining a compound of formula I with a binder, the resulting formulation leads to several advantages. For example, since the compounds of formula I are soluble they may be deposited in a liquid form, for example from solution. With the additional use of the binder the formulation can be coated onto a large area in a highly uniform manner. Furthermore, when a binder is used in the formulation it is possible to control the properties of the formulation to adjust to printing processes, for example viscosity, solid content, surface tension. Whilst not wishing to be bound by any particular theory it is also anticipated that the use of a binder in the formulation fills in volume between crystalline grains otherwise being void, making the organic semiconducting layer less sensitive to air and moisture. For example, layers formed according to the process of the present invention show very good stability in OFET devices in air.

The invention also provides an organic semiconducting layer which comprises the organic semiconducting layer formulation.

The invention further provides a process for preparing an organic semiconducting layer, said process comprising the following steps:
(i) depositing on a substrate a liquid layer of a formulation comprising one or more compounds of formula I as described above and below, one or more organic binder resins or precursors thereof, and optionally one or more solvents,
(ii) forming from the liquid layer a solid layer which is the organic semiconducting layer,
(iii) optionally removing the layer from the substrate.

The process is described in more detail below.

The invention additionally provides an electronic device comprising the said organic semiconducting layer. The electronic device may include, without limitation, an organic field effect transistor (OFET), organic light emitting diode (OLED), photodetector, sensor, logic circuit, memory element, capacitor or photovoltaic (PV) cell. For example, the active semiconductor channel between the drain and source in an OFET may comprise the layer of the invention. As another example, a charge (hole or electron) injection or transport layer in an OLED device may comprise the layer of the invention. The formulations according to the present invention and layers formed therefrom have particular utility in OFETs especially in relation to the preferred embodiments described herein.

In a preferred embodiment of the present invention the semiconducting compound of formula I has a charge carrier mobility, μ, of more than 10⁻⁵ cm²V⁻¹s⁻¹, preferably of more than 10⁻⁴ cm²V⁻¹s⁻¹, more preferably of more than 10⁻³ cm²V⁻¹s⁻¹, still more preferably of more than 10⁻² cm²V⁻¹s⁻¹ and most preferably of more than 10⁻¹ cm²V⁻¹s⁻¹.

The binder, which is typically a polymer, may comprise either an insulating binder or a semiconducting binder, or mixtures thereof may be referred to herein as the organic binder, the polymeric binder or simply the binder.

Preferred binders according to the present invention are materials of low permittivity, that is, those having a permittivity ∈ at 1,000 Hz of 3.3 or less. The organic binder preferably has a permittivity ∈ at 1,000 Hz of 3.0 or less, more preferably 2.9 or less. Preferably the organic binder has a permittivity ∈ at 1,000 Hz of 1.7 or more. It is especially preferred that the permittivity of the binder is in the range from 2.0 to 2.9. Whilst not wishing to be bound by any particular theory it is believed that the use of binders with a permittivity ∈ of greater than 3.3 at 1,000 Hz, may lead to a reduction in the OSC layer mobility in an electronic device, for example an OFET. In addition, high permittivity binders could also result in increased current hysteresis of the device, which is undesirable.

An example of a suitable organic binder is polystyrene. Further examples are given below.

In one type of preferred embodiment, the organic binder is one in which at least 95%, more preferably at least 98% and especially all of the atoms consist of hydrogen, fluorine and carbon atoms.

It is preferred that the binder normally contains conjugated bonds, especially conjugated double bonds and/or aromatic rings.

The binder should preferably be capable of forming a film, more preferably a flexible film. Polymers of styrene and α-methyl styrene, for example copolymers including styrene, α-methylstyrene and butadiene may suitably be used.

Binders of low permittivity of use in the present invention have few permanent dipoles which could otherwise lead to random fluctuations in molecular site energies. The permittivity ∈ (dielectric constant) can be determined by the ASTM D150 test method.

It is also preferred that in the present invention binders are used which have solubility parameters with low polar and hydrogen bonding contributions as materials of this type have low permanent dipoles. A preferred range for the solubility parameters (‘Hansen parameter’) of a binder for use in accordance with the present invention is provided in Table 1 below.

TABLE 1

| | Hansen parameter | | |
|---|---|---|---|
| | δ_{d} MPa^{1/2} | δₚ MPa^{1/2} | δₕ MPa^{1/2} |
| Preferred range | 14.5+ | 0-10 | 0-14 |
| More preferred range | 16+ | 0-9 | 0-12 |
| Most preferred range | 17+ | 0-8 | 0-10 |

The three dimensional solubility parameters listed above include: dispersive (δ_{d}), polar (δₚ and hydrogen bonding (δₕ) components (C. M. Hansen, Ind. Eng. and Chem., Prod. Res. and Devl., 9, No 3, p 282., 1970). These parameters may be determined empirically or calculated from known molar group contributions as described in Handbook of Solubility Parameters and Other Cohesion Parameters ed. A. F. M. Barton, CRC Press, 1991. The solubility parameters of many known polymers are also listed in this publication.

It is desirable that the permittivity of the binder has little dependence on frequency. This is typical of non-polar materials. Polymers and/or copolymers can be chosen as the binder by the permittivity of their substituent groups. A list of suitable and preferred low polarity binders is given (without limiting to these examples) in Table 2:

TABLE 2

| | typical low |
|---|---|
| | frequency |
| Binder | permittivity (ε) |
| polystyrene | 2.5 |
| poly(α-methylstyrene) | 2.6 |
| poly(α-vinylnaphtalene) | 2.6 |
| poly(vinyltoluene) | 2.6 |
| polyethylene | 2.2-2.3 |
| cis-polybutadiene | 2.0 |
| polypropylene | 2.2 |
| polyisoprene | 2.3 |
| poly(4-methyl-1-pentene) | 2.1 |
| poly (4-methylstyrene) | 2.7 |
| poly(chorotrifluoroethylene) | 2.3-2.8 |
| poly(2-methyl-1,3-butadiene) | 2.4 |
| poly(p-xylylene) | 2.6 |
| poly(α-α-α′-α′ tetrafluoro-p-xylylene) | 2.4 |
| poly[1,1-(2-methyl propane)bis(4-phenyl)carbonate] | 2.3 |
| poly(cyclohexyl methacrylate) | 2.5 |
| poly(chlorostyrene) | 2.6 |
| poly(2,6-dimethyl-1,4-phenylene ether) | 2.6 |
| polyisobutylene | 2.2 |
| poly(vinyl cyclohexane) | 2.2 |
| poly(vinylcinnamate) | 2.9 |
| poly(4-vinylbiphenyl) | 2.7 |

Other polymers suitable as binders include poly(1,3-butadiene) or polyphenylene.

Especially preferred are formulations wherein the binder is selected from poly-α-methyl styrene, polystyrene and polytriarylamine or any copolymers of these, and the solvent is selected from xylene(s), toluene, tetralin and cyclohexanone.

Copolymers containing the repeat units of the above polymers are also suitable as binders. Copolymers offer the possibility of improving compatibility with the compounds of formula I, modifying the morphology and/or the glass transition temperature of the final layer composition. It will be appreciated that in the above table certain materials are insoluble in commonly used solvents for preparing the layer. In these cases analogues can be used as copolymers. Some examples of copolymers are given in Table 3 (without limiting to these examples). Both random or block copolymers can be used. It is also possible to add some more polar monomer components as long as the overall composition remains low in polarity.

TABLE 3

| | typical low |
|---|---|
| | frequency |
| Binder | permittivity (ε) |
| poly(ethylene/tetrafluoroethylene) | 2.6 |
| poly(ethylene/chlorotrifluoroethylene) | 2.3 |
| fluorinated ethylene/propylene copolymer | 2-2.5 |
| polystyrene-co-α-methylstyrene | 2.5-2.6 |
| ethylene/ethyl acrylate copolymer | 2.8 |
| poly(styrene/10% butadiene) | 2.6 |
| poly(styrene/15% butadiene) | 2.6 |
| poly(styrene/2,4 dimethylstyrene) | 2.5 |
| Topas ™ (all grades) | 2.2-2.3 |

Other copolymers may include: branched or non-branched polystyrene-block-polybutadiene, polystyrene-block(polyethylene-ran-butylene)-block-polystyrene, polystyrene-block-polybutadiene-block-polystyrene, polystyrene-(ethylene-propylene)-diblock-copolymers (e.g. KRATON®-G1701E, Shell), poly(propylene-co-ethylene) and poly(styrene-co-methylmethacrylate).

Preferred insulating binders for use in the organic semiconductor layer formulation according to the present invention are poly(α-methylstyrene), polyvinylcinnamate, poly(4-vinylbiphenyl), poly(4-methylstyrene), and Topas™ 8007 (linear olefin, cyclo-olefin(norbornene) copolymer available from Ticona, Germany). Most preferred insulating binders are poly(α-methylstyrene), polyvinylcinnamate and poly(4-vinylbiphenyl).

The binder can also be selected from crosslinkable binders, like e.g. acrylates, epoxies, vinylethers, thiolenes etc., preferably having a sufficiently low permittivity, very preferably of 3.3 or less. The binder can also be mesogenic or liquid crystalline.

As mentioned above the organic binder may itself be a semiconductor, in which case it will be referred to herein as a semiconducting binder. The semiconducting binder is still preferably a binder of low permittivity as herein defined. Semiconducting binders for use in the present invention preferably have a number average molecular weight (Mₙ) of at least 1500-2000, more preferably at least 3000, even more preferably at least 4000 and most preferably at least 5000. The semiconducting binder preferably has a charge carrier mobility, μ, of at least 10⁻⁵ cm²V⁻¹s⁻¹, more preferably at least 10⁻⁴ cm²V⁻¹s⁻¹.

A preferred class of semiconducting binder is a polymer as disclosed in U.S. Pat. No. 6,630,566, preferably an oligomer or polymer having repeat units of formula 1:

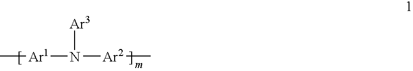

wherein
Ar¹, Ar² and Ar³ which may be the same or different, denote, independently if in different repeat units, an optionally substituted aromatic group that is mononuclear or polynuclear, and
m is an integer ≧1, preferably ≧6, preferably ≧10, more preferably ≧15 and most preferably ≧20.

In the context of Ar¹, Ar² and Ar³, a mononuclear aromatic group has only one aromatic ring, for example phenyl or phenylene. A polynuclear aromatic group has two or more aromatic rings which may be fused (for example napthyl or naphthylene), individually covalently linked (for example biphenyl) and/or a combination of both fused and individually linked aromatic rings. Preferably each Ar¹, Ar² and Ar³ is an aromatic group which is substantially conjugated over substantially the whole group.

Further preferred classes of semiconducting binders are those containing substantially conjugated repeat units. The semiconducting binder polymer may be a homopolymer or copolymer (including a block-copolymer) of the general formula 2:

A_{(c)}B_{(d)} . . . Z_{(z)} 2

wherein A, B, . . . , Z each represent a monomer unit and (c), (d), . . . (z) each represent the mole fraction of the respective monomer unit in the polymer, that is each (c), (d), . . . (z) is a value from 0 to 1 and the total of (c)+(d)+ . . . + (z)=1.

Examples of suitable and preferred monomer units A, B, . . . Z include units of formula 1 above and of formulae 3 to 8 given below (wherein m is as defined in formula 1:

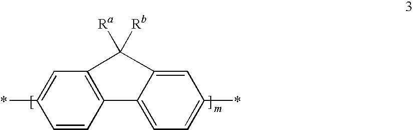

wherein
R^{a} and R^{b} are independently of each other selected from H, F, CN, NO₂, —N(R^{c})(R^{d}) or optionally substituted alkyl, alkoxy, thioalkyl, acyl, aryl,
R^{c} and R^{d} are independently or each other selected from H, optionally substituted alkyl, aryl, alkoxy or polyalkoxy or other substituents,
   and wherein the asterisk (*) is any terminal or end capping group including H, and the alkyl and aryl groups are optionally fluorinated;

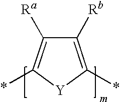

wherein
Y is Se, Te, O, S or —N(R^{e}), preferably O, S or —N(R^{e})—,
R^{e} is H, optionally substituted alkyl or aryl,
R^{a} and R^{b} are as defined in formula 3;

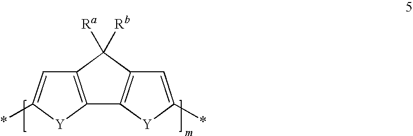

wherein R^{a}, R^{b} and Y are as defined in formulae 3 and 4;

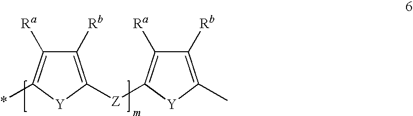

wherein R^{a}, R^{b} and Y are as defined in formulae 3 and 4,
Z is —C(T¹)=C(T²)-, —C≡C—, —N(R^{f})—, —N═N—, (R^{f})═N—, —N═C(R^{f})—,
T¹ and T² independently of each other denote H, Cl, F, —CN or lower alkyl with 1 to 8 C atoms,
R^{f} is H or optionally substituted alkyl or aryl;

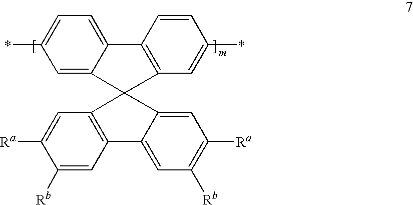

wherein R^{a} and R^{b} are as defined in formula 3;

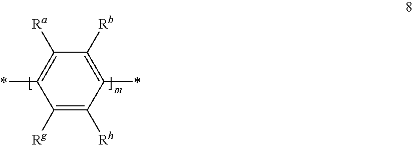

wherein R^{a}, R^{b}, R^{g} and R^{h} independently of each other have one of the meanings of R^{a} and R^{b} in formula 3.

In the case of the polymeric formulae described herein, such as formulae 1 to 8, the polymers may be terminated by any terminal group, that is any end-capping or leaving group, including H.

In the case of a block-copolymer, each monomer A, B, . . . Z may be a conjugated oligomer or polymer comprising a number, for example 2 to 50, of the units of formulae 3-8. The semiconducting binder preferably includes: arylamine, fluorene, thiophene, Spiro bifluorene and/or optionally substituted aryl (for example phenylene) groups, more preferably arylamine, most preferably triarylamine groups. The aforementioned groups may be linked by further conjugating groups, for example vinylene.

In addition, it is preferred that the semiconducting binder comprises a polymer (either a homo-polymer or copolymer, including block-copolymer) containing one or more of the aforementioned arylamine, fluorene, thiophene and/or optionally substituted aryl groups. A preferred semiconducting binder comprises a homo-polymer or copolymer (including block-copolymer) containing arylamine (preferably triarylamine) and/or fluorene units. Another preferred semiconducting binder comprises a homo-polymer or co-polymer (including block-copolymer) containing fluorene and/or thiophene units.

The semiconducting binder may also contain carbazole or stilbene repeat units. For example polyvinylcarbazole or polystilbene polymers or copolymers may be used. The semiconducting binder may optionally contain DBBDT segments (for example repeat units as described for formula I above) to improve compatibility with the soluble compounds of formula.

The most preferred semiconducting binders for use in the organic semiconductor layer formulation according to the present invention are poly(9-vinylcarbazole) and PTAA1, a polytriarylamine of the following formula

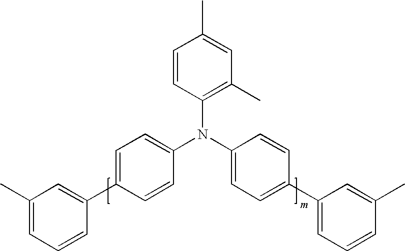

wherein m is as defined in formula 1.

For application of the semiconducting layer in p-channel FETs, it is desirable that the semiconducting binder should have a higher ionisation potential than the semiconducting compound of formula I, otherwise the binder may form hole traps. In n-channel materials the semiconducting binder should have lower electron affinity than the n-type semiconductor to avoid electron trapping.

The formulation according to the present invention may be prepared by a process which comprises:
(i) first mixing a compound of formula I and an organic binder or a precursor thereof. Preferably the mixing comprises mixing the two components together in a solvent or solvent mixture,
(ii) applying the solvent(s) containing the compound of formula I and the organic binder to a substrate; and optionally evaporating the solvent(s) to form a solid organic semiconducting layer according to the present invention,
(iii) and optionally removing the solid layer from the substrate or the substrate from the solid layer.

In step (i) the solvent may be a single solvent or the compound of formula I and the organic binder may each be dissolved in a separate solvent followed by mixing the two resultant solutions to mix the compounds.

The binder may be formed in situ by mixing or dissolving a compound of formula I in a precursor of a binder, for example a liquid monomer, oligomer or crosslinkable polymer, optionally in the presence of a solvent, and depositing the mixture or solution, for example by dipping, spraying, painting or printing it, on a substrate to form a liquid layer and then curing the liquid monomer, oligomer or crosslinkable polymer, for example by exposure to radiation, heat or electron beams, to produce a solid layer. If a preformed binder is used it may be dissolved together with the compound of formula I in a suitable solvent, and the solution deposited for example by dipping, spraying, painting or printing it on a substrate to form a liquid layer and then removing the solvent to leave a solid layer. It will be appreciated that solvents are chosen which are able to dissolve both the binder and the compound of formula I, and which upon evaporation from the solution blend give a coherent defect free layer.

Suitable solvents for the binder or the compound of formula I can be determined by preparing a contour diagram for the material as described in ASTM Method D 3132 at the concentration at which the mixture will be employed. The material is added to a wide variety of solvents as described in the ASTM method.

It will also be appreciated that in accordance with the present invention the formulation may also comprise two or more compounds of formula I and/or two or more binders or binder precursors, and that the process for preparing the formulation may be applied to such formulations.

Examples of suitable and preferred organic solvents include, without limitation, dichloromethane, trichloromethane, monochlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetralin, decalin, indane and/or mixtures thereof.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 38, No 496, 296 (1966)”. Solvent blends may also be used and can be identified as described in “Solvents, W.H.Ellis, Federation of Societies for Coatings Technology, p 9-10, 1986”. Such a procedure may lead to a blend of ‘non’ solvents that will dissolve both the binder and the compound of formula I, although it is desirable to have at least one true solvent in a blend.

Especially preferred solvents for use in the formulation according to the present invention, with insulating or semiconducting binders and mixtures thereof, are xylene(s), toluene, tetralin and o-dichlorobenzene.

The proportions of binder to the compound of formula I in the formulation or layer according to the present invention are typically 20:1 to 1:20 by weight, preferably 10:1 to 1:10 more preferably 5:1 to 1:5, still more preferably 3:1 to 1:3 further preferably 2:1 to 1:2 and especially 1:1. Surprisingly and beneficially, dilution of the compound of formula I in the binder has been found to have little or no detrimental effect on the charge mobility, in contrast to what would have been expected from the prior art.

In accordance with the present invention it has further been found that the level of the solids content in the organic semiconducting layer formulation is also a factor in achieving improved mobility values for electronic devices such as OFETs. The solids content of the formulation is commonly expressed as follows:

$$Solids content ( \% ) = \frac{a + b}{a + b + c} \times 100$$

wherein
a=mass of compound of formula I, b=mass of binder and c=mass of solvent.

The solids content of the formulation is preferably 0.1 to 10% by weight, more preferably 0.5 to 5% by weight.

Surprisingly and beneficially, dilution of the compound of formula I in the binder has been found to have little or no effect on the charge mobility, in contrast to what would have been expected from the prior art.

It is desirable to generate small structures in modern microelectronics to reduce cost (more devices/unit area), and power consumption. Patterning of the layer of the invention may be carried out by photolithography or electron beam lithography.

Liquid coating of organic electronic devices such as field effect transistors is more desirable than vacuum deposition techniques. The formulations of the present invention enable the use of a number of liquid coating techniques. The organic semiconductor layer may be incorporated into the final device structure by, for example and without limitation, dip coating, spin coating, ink jet printing, letter-press printing, screen printing, doctor blade coating, roller printing, reverse-roller printing, offset lithography printing, flexographic printing, web printing, spray coating, brush coating or pad printing. The present invention is particularly suitable for use in spin coating the organic semiconductor layer into the final device structure.

Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the mixture of the compound of formula I and the binder should be first dissolved in a suitable solvent. Solvents must fulfil the requirements stated above and must not have any detrimental effect on the chosen print head. Additionally, solvents should have boiling points >100° C., preferably >140° C. and more preferably >150° C. in order to prevent operability problems caused by the solution drying out inside the print head. Suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a formulation according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the binder and the compound of formula I which reduces or prevents clogging of the jets and separation of the components during spraying. The solvent(s) may include those selected from the following list of examples: dodecylbenzene, 1-methyl-4-tert-butylbenzene, terpineol limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The use of the binder in the present invention also allows the viscosity of the coating solution to be tuned to meet the requirements of the particular print head.

The semiconducting layer of the present invention is typically at most 1 micron (=1 μm) thick, although it may be thicker if required. The exact thickness of the layer will depend, for example, upon the requirements of the electronic device in which the layer is used. For use in an OFET or OLED, the layer thickness may typically be 500 nm or less.

In the semiconducting layer of the present invention there may be used two or more different compounds of formula I. Additionally or alternatively, in the semiconducting layer there may be used two or more organic binders of the present invention.

As mentioned above, the invention further provides a process for preparing the organic semiconducting layer which comprises (i) depositing on a substrate a liquid layer of a formulation which comprises one or more compounds of formula I, one or more organic binders or precursors thereof and optionally one or more solvents, and (ii) forming from the liquid layer a solid layer which is the organic semiconducting layer.

In the process, the solid layer may be formed by evaporation of the solvent and/or by reacting the binder resin precursor (if present) to form the binder resin in situ. The substrate may include any underlying device layer, electrode or separate substrate such as silicon wafer or polymer substrate for example.

In a particular embodiment of the present invention, the binder may be alignable, for example capable of forming a liquid crystalline phase. In that case the binder may assist alignment of the compound of formula I, for example such that their aromatic core is preferentially aligned along the direction of charge transport. Suitable processes for aligning the binder include those processes used to align polymeric organic semiconductors and are described in prior art, for example in WO 03/007397 (Plastic Logic).

The formulation according to the present invention can additionally comprise one or more further components like for example surface-active compounds, lubricating agents, wetting agents, dispersing agents, hydrophobing agents, adhesive agents, flow improvers, defoaming agents, deaerators, diluents, reactive or non-reactive diluents, auxiliaries, colourants, dyes or pigments, furthermore, especially in case crosslinkable binders are used, catalysts, sensitizers, stabilizers, inhibitors, chain-transfer agents or co-reacting monomers.

The present invention also provides the use of the semiconducting compound, formulation or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising the formulation according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The compound or formulation may be used, for example as a layer or film, in a field effect transistor (FET) for example as the semiconducting channel, organic light emitting diode (OLED) for example as a hole or electron injection or transport layer or electroluminescent layer, photodetector, chemical detector, photovoltaic cell (PVs), capacitor sensor, logic circuit, display, memory device and the like. The compound or formulation may also be used in electrophotographic (EP) apparatus.

The compound or formulation is preferably solution coated to form a layer or film in the aforementioned devices or apparatus to provide advantages in cost and versatility of manufacture. The improved charge carrier mobility of the compound or formulation of the present invention enables such devices or apparatus to operate faster and/or more efficiently. The compound, formulation and layer of the present invention are especially suitable for use in an organic field effect transistor OFET as the semiconducting channel. Accordingly, the invention also provides an organic field effect transistor (OFET) comprising a gate electrode, an insulating (or gate insulator) layer, a source electrode, a drain electrode and an organic semiconducting channel connecting the source and drain electrodes, wherein the organic semiconducting channel comprises an organic semiconducting layer according to the present invention. Other features of the OFET are well known to those skilled in the art.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.
   wherein the semiconductor layer preferably comprises a compound of formula I, very preferably a formulation comprising a compound of formula I and an organic binder as described above and below.

The OFET device can be a top gate device or a bottom gate device. Suitable structures and manufacturing methods of an OFET device are known to the skilled in the art and are described in the literature, for example in WO 03/052841.

The gate insulator layer preferably comprises a fluoropolymer, like e.g. the commercially available Cytop 809M® or Cytop 107M® (from Asahi Glass).

Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377).

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention.

Independent protection may be sought for these features in addition to or alternative to any invention presently claimed.

The invention will now be described in more detail by reference to the following examples, which are illustrative only and do not limit the scope of the invention.

### Example 1

6,12-bis(triethylsilylethynyl)-dibenzo[d,d′]-benzo[1,2-b;4,5-b′]dithiophene (1) is prepared as described below

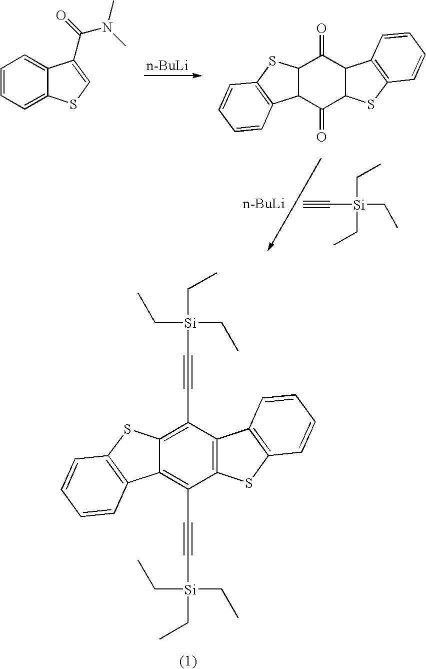

### Step 1-1: Dibenzo[d,d′]benzo[1,2-b:4,5-b′]dithiophene-6,12-dione

Benzo[b]thiophene-3-carboxylic acid dimethylamide (4.27 g, 20.8 mmol) is dissolved in anhydrous diethyl ether (150 ml) then cooled to −78° C., followed by the slow addition of n-BuLi (1.6 M in hexanes, 13.5 ml, 21.6 mmol). After complete addition, the reaction mixture is allowed to warm to room temperature and stirred for 1 h, then terminated by addition of water. The precipitate is collected by filtration and washed with water and ether, to give product as a red solid (1.73 g, 52%). ¹H NMR (300 MHz, CDCl₃): δ 7.06 (d, J=8.1 Hz, 2H, Ar—H), 6.37 (d, J=7.3 Hz, 2H, Ar—H), 5.86 (m, 4H, Ar—H); MS: (m/e) 320 (M⁺, 100), 292, 264, 219, 132.

### Step 1-2: 6,12-bis(triethylsilylethynyl)-dibenzo[d,d′]benzo[1,2-b;4,5-b′]dithiophene

To a solution of triethylsilyacetylene (3.62 g, 25.8 mmol) in dioxane is added n-BuLi (2.5 M in hexanes, 10.3 ml, 25.8 mmol) dropwise at room temperature. This solution is stirred for 30 min, followed by the addition of dibenzo[d,d′]benzo[1,2-b;4,5-b′]dithiophene-6,12-dione (1.65 g, 5.2 mmol) The resulting mixture is heated at reflux for 3 h. After the reaction mixture is cooled to room temperature, solid SnCl₂ (5 g) and conc. HCl solution (10 ml) is added, and the resultant mixture is stirred for 30 min at room temperature. The precipitate is collected by filtration and washed with water to give a yellow solid, which is recrystallised with acetone to give yellow crystals (2.13 g, 73%). ¹H NMR (300 MHz, CDCl₃): δ 9.30 (dd, J=7.4 and 1.5 Hz, 2H, Ar—H), 7.92 (dd, J=7.2 and 1.3 Hz, 2H, Ar—H), 7.51 (m, 4H, Ar—H), 1.22 (t, J=7.9 Hz, 18H, CH₃), 0.88 (q, J=7.9 Hz, 12H, CH₂); ¹³C NMR (75 MHz, CDCl₃): δ 142.9, 140.3, 135.3, 132.5, 127.3, 124.9, 124.2, 122.6, 112.2, 107.1, 102.4, 7.74, 4.51; MS (m/e): 566 (M⁺, 100), 509, 481, 198, 87.

The single crystal packing of compound 1 is examined by XRD of single crystal grown from THF/acetonitrile. Compound 1 exhibits a herringbone motif with close intramolecular contacts.

### Example 2

6,12-bis(triisopropylsilylethynyl)-dibenzo[d,d′]benzo[1,2-b;4,5-b′]dithiophene (2) is prepared as described below

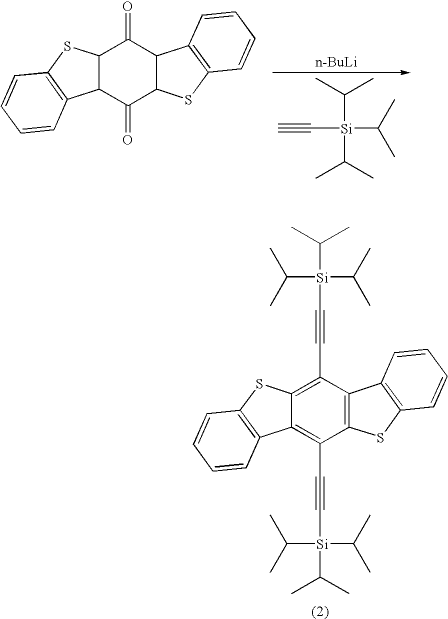

To a solution of triisopropylsilyacetylene (1.34 g, 7.3 mmol) in dioxane is added n-BuLi (1.6 M in hexanes, 4.5 ml, 7.2 mmol) dropwise at room temperature. This solution is stirred for 30 min, followed by the addition of dibenzo[d,d′]benzo[1,2-b;4,5-b′]dithiophene-6,12-dione (0.47 g, 1.5 mmol). The resulting mixture is heated at reflux for 3.5 h. After the reaction mixture is cooled to room temperature, solid SnCl₂ (˜3 g) and conc. HCl solution (10 ml) is added, and the resultant mixture is stirred for about 1 h at room temperature. The precipitate is collected by filtration and washed with water and acetone to give a yellow solid, which is recrystallised with acetone to give yellow crystals (0.43 g, 45%). ¹H NMR (300 MHz, CDCl₃): δ 9.38 (dd, J=7.2 and 1.2 Hz, 2H, Ar—H), 7.93 (dd, J=7.5 and 1.0 Hz, 2H, Ar—H), 7.49 (m, 4H, Ar—H), 1.30 (m, 42H, CH₃ and CH); ¹³C NMR (75 MHz, CDCl₃): δ 143.1, 140.3, 135.3, 132.6, 127.3, 125.0, 124.2, 122.6, 112.3, 106.1, 103.2, 18.9, 11.5.

### Example 3

6,12-bis(trimethylsilylethynyl)-dibenzo[d,d′]benzo[1,2-b;4,5-b′]dithiophene (3) is prepared as described below

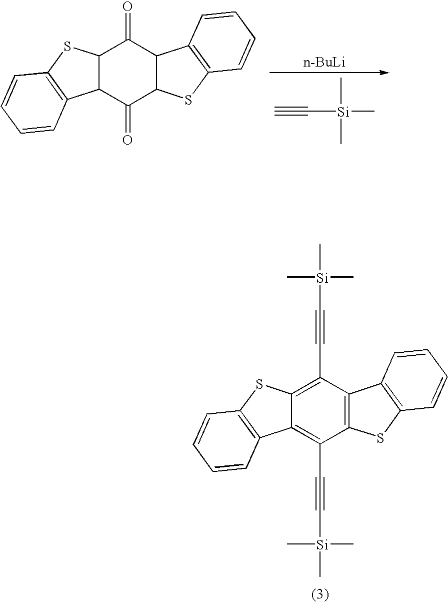

To a solution of trimethylsilyacetylene (0.35 g, 3.6 mmol) in dioxane is added n-BuLi (1.6 M in hexanes, 2.20 ml, 3.5 mmol) dropwise at room temperature. This solution is stirred for 30 min, followed by the addition of dibenzo[d,d′]benzo[1,2-b;4,5-b′]dithiophene-6,12-dione (0.22 g, 0.7 mmol) The resulting mixture is heated at reflux for 3 h. After the reaction mixture is cooled to room temperature, solid SnCl₂ (2 g) and conc. HCl solution (7 ml) is added, and the resultant mixture is stirred for 30 min at room temperature. The precipitate is collected by filtration and washed with water to give a yellow solid. This is purified by column chromatography, eluting with petrol/ethyl acetate (from 10:0 to 9:1), to give a yellow solid, which is recrystallised with THF/petrol (1:10) to give yellow crystals (0.14 g, 42%). ¹H NMR (300 MHz, CDCl₃): δ 9.18 (dd, J=7.0 and 1.3 Hz, 2H, Ar—H), 7.90 (dd, J=7.5 and 1.1 Hz, 2H, Ar—H), 7.50 (m, 4H, Ar—H), 0.47 (s, 18H, CH₃); ¹³C NMR (75 MHz, CDCl₃): δ 142.8, 140.4, 135.3, 132.5, 127.4, 124.8, 124.3, 122.6, 112.1, 109.1, 101.4, 0.01.

### Example 4

2,8-Bis(phenylvinyl)-6,12-bis(triethylsilylethynyl)-dibenzo[d,d′]benzo[1,2-b;4,5-b′]dithiophene (4) is prepared as described below

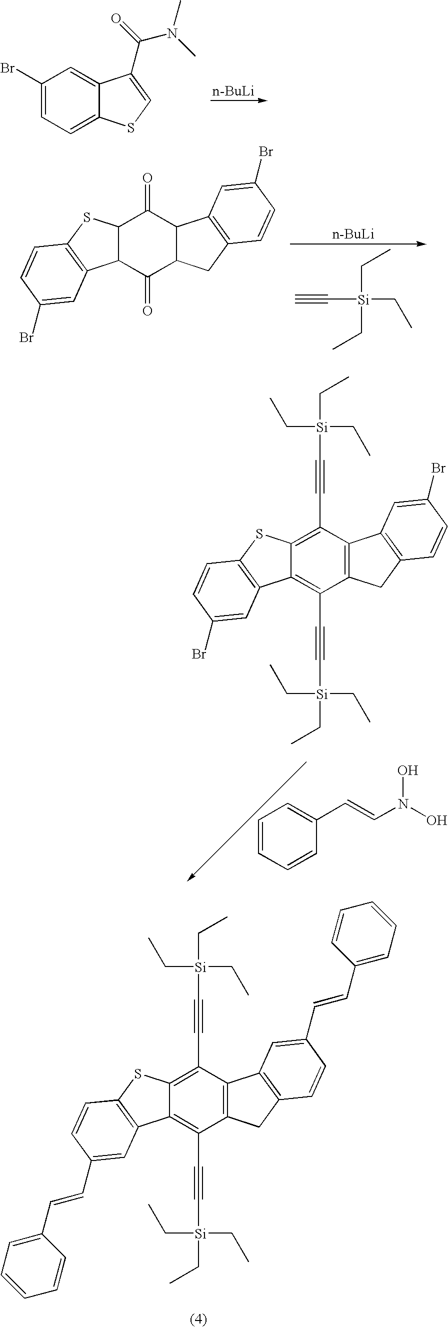

### Step 4-1: 2,8-dibromo-dibenzo[d,d′]benzo[1,2-b;4,5-b′]dithiophene-6,12-dione

5-Bromobenzo[b]thiophene-3-carboxylic acid dimethylamide (5.0 g, 20.8 mmol) was dissolved in anhydrous diethyl ether (150 ml), followed by the slow addition of n-BuLi (1.6 M in hexanes, 13.5 ml, 21.6 mmol). After complete addition, the reaction mixture was allowed to warm to room temperature and stirred for 1 h, then terminated by addition of water. The precipitate was collected by filtration and washed with water and ether, to give product as a red solid (1.85 g, 44%). MS: (m/e) 480 (M⁺), 478 (M⁺), 398, 400 281, 253, 207 (100); IR: v (cm⁻¹) 1651 (C═O).

### Step 4-2: 2,8-dibromo-6,12-bis(triethylsilylethynyl)-dibenzo[d,d′]benzo[1,2-b:4,5-b′]dithiophene

To a solution of triethylsilyacetylene (2.8 g, 20.0 mmol) in dioxane (80 ml) was added n-BuLi (1.6 M in hexanes, 10.5 ml, 16.8 mmol) dropwise at room temperature. This solution was stirred for 30 min, followed by the addition of 2,8-dibromodibenzo[d,d′]benzo[1,2-b;4,5-b′]dithiophene-6,12-dione (1.83 g, 3.8 mmol). The resulting mixture was heated at reflux for 3 h. After the reaction mixture was cooled to room temperature, solid SnCl₂ (5 g) and conc. HCl solution (10 ml) was added, and the resultant mixture was stirred for 30 min at room temperature. The precipitate was collected by filtration and washed with water to give a yellow solid, which was recrystallised with acetone to give yellow crystals (2.45 g, 88%). ¹H NMR (300 MHz, CDCl₃): δ9.39 (d, J=1.8 Hz, 2H, Ar—H), 7.78 (d, J=8.5 Hz, 2H, Ar—H), 7.62 (dd, J=8.5 and 1.8 Hz, 2H, Ar—H), 1.23 (t, J=7.8 Hz, 18H, CH₃), 0.91 (q, J=7.8 Hz, 12H, CH₂); ¹³C NMR (75 MHz, CDCl₃): δ 143.3, 139.0, 136.7, 131.5, 130.4, 127.5, 123.8, 118.4, 112.4, 108.3, 101.5, 7.9, 4.4; IR: v (cm⁻¹) 2150 (C≡C).

### Step 4-3: 2,8-Bis(phenylvinyl)-6,12-bis(triethylsilylethynyl)-dibenzo[d,d′]benzo-[1,2-b;4,5-b′]dithiophene

2,8-Dibromo-6,12-bis(triethylsilylethynyl)-dibenzo[d,d′]benzo[1,2-b;4,5-b′]dithiophene (0.72 g, 0.99 mmol) was dissolved in dry THF (12 ml) in a 20 ml microwave vial, followed by the addition of tetrakis(triphenyl-phosphine)-palladium(0) (0.1 g). The mixture was stirred for 5 min, then trans-2-phenylvinylboronic acid (0.32 g, 2.16 mmol) and potassium carbonate solution (1.2 g of K₂CO₃ was dissolved in 3 ml water) were added. The resultant mixture was degassed with N₂ for 5 min, then placed in microwave reactor and heated at 100° C. for 2 min, 120° C. for 2 min and 140° C. for 20 min. After cooling, the mixture was poured into water and the precipitate was collected by filtration, washed with water to give a brown solid. This solid was purified by column chromatography, eluting with ethyl acetate, to give a yellow solid, which was recrystallised with acetone/THF to give yellow crystals (0.43 g, 56%). ¹H NMR (300 MHz, CDCl₃): δ9.27 (d, J=1.5 Hz, 2H, Ar—H), 7.88 (d, J=8.3 Hz, 2H, Ar—H), 7.75 (dd, J=8.3 and 1.5 Hz, 2H, Ar—H), 7.16-7.56 (m, 14H, ═CH and Ar—H), 1.25 (t, J=7.5 Hz, 18H, CH₃), 0.96 (q, J=7.5 Hz, 12H, CH₂); ¹³C NMR (75 MHz, CDCl₃): δ 143.6, 139.6, 137.5, 135.8, 134.1, 132.3, 128.9, 128.8, 128.5, 127.6, 126.4, 125.0, 123.5, 122.7, 112.3, 107.2, 102.4, 7.9, 4.6.

### Example 5

2,8-Bis[(5′-methyl)thioenyl]-6,12-bis(triethylsilylethynyl)-dibenzo[d,d′]benzo[1,2-b;4,5-b′]dithiophene (5) is prepared as described below:

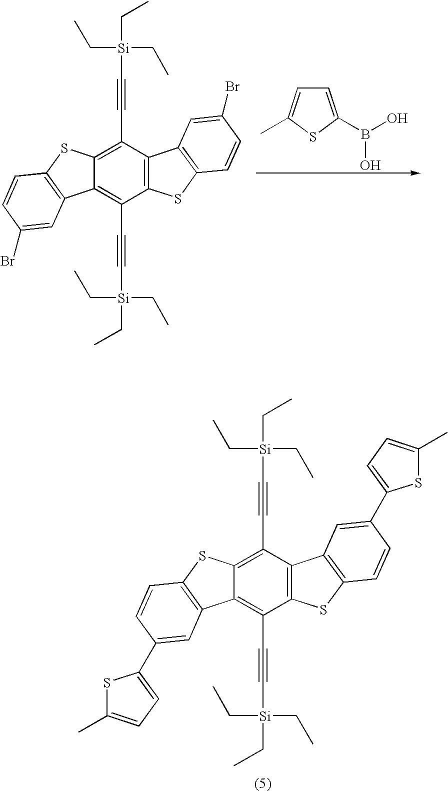

### Step 5.1 2,8-Bis[(5′-methyl)thioenyl]-6,12-bis(triethylsilylethynyl)-dibenzo[d,d′]benzo[1,2-b;4,5-b′]dithiophene

2,8-Dibromo-6,12-bis(triethylsilylethynyl)-dibenzo[d,d′]benzo[1,2-b;4,5-b′]dithio-phene (0.53 g, 0.73 mmol) was dissolved in dry THF (10 ml) in a 20 ml microwave vial, followed by the addition of tetrakis(triphenylphosphine)palladium(0) (0.1 g). The mixture was stirred for 5 min, then 5-methyl-2-thiopheneboronic acid (0.36 g, 1.61 mmol) and potassium carbonate solution (0.9 g of K₂CO₃ was dissolved in 3 ml water) were added. The resultant mixture was degassed with N₂ for 5 min, then placed in microwave reactor and heated at 100° C. for 2 min, 120° C. for 2 min and 140° C. for 20 min. After cooling, the mixture was poured into water and the precipitate was collected by filtration, washed with water to give a brown solid. This solid was purified by column chromatography, eluting with ethyl acetate, to give a yellow solid, which was recrystallised with acetone/THF to give brown crystals (0.23 g, 41%). ¹H NMR (300 MHz, CDCl₃): δ 9.41 (d, J=1.5 Hz, 2H, Ar—H), 7.89 (d, J=8.3 Hz, 2H, Ar—H), 7.72 (dd, J=8.3 and 1.5 Hz, 2H, Ar—H), 7.21 (d, J=3.6 Hz, 2H, Ar—H), 6.77 (dd, J=3.6 and 0.9 Hz, 2H, Ar—H), 1.20 (t, J=7.5 Hz, 18H, CH3), 0.95 (q, J=7.5 Hz, 12H, CH2); ¹³C NMR (75 MHz, CDCl₃): δ 144.0, 142.1, 139.5, 138.9, 135.9, 132.2, 131.5, 126.1, 125.4, 123.2, 122.8, 121.7, 112.4, 107.7, 102.3, 7.8, 4.6.

### Example 6

The transistor properties of OFETs comprising compounds 1-5 are measured as follows:

A test field effect transistor is manufactured by using a PEN substrate upon which are patterned Pt/Pd source and drain electrodes by standard techniques, for example shadow masking. The devices are fabricated by spin coating a 4 wt % blend of each of compounds (1-5), respectively, in 1:1 mixture with poly(triaryl)amine in tetralin, followed by drying at 100° C. for 30 seconds on a hotplate. The insulator material (Cytop 809M®, a formulation of a fluoropolymer in a fluorosolvent, available from Asahi Glass) is spin-coated onto the semiconductor giving a thickness typically of approximately 1 μm. The samples are placed once more in an oven at 100° C. for 20 minutes to evaporate solvent from the insulator. A gold gate contact is defined over the device channel area by evaporation through a shadow mask. To determine the capacitance of the insulator layer a number of devices are prepared which consist of a non-patterned Pt/Pd base layer, an insulator layer prepared in the same way as that on the FET device, and a top electrode of known geometry. The capacitance is measured using a hand-held multimeter, connected to the metal either side of the insulator. Other defining parameters of the transistor are the length of the drain and source electrodes facing each other (W=30 mm) and their distance from each other (L=130 μm).

The voltages applied to the transistor are relative to the potential of the source electrode. In the case of a p-type gate material, when a negative potential is applied to the gate, positive charge carriers (holes) are accumulated in the semiconductor on the other side of the gate dielectric. (For an n-channel FET, positive voltages are applied). This is called the accumulation mode. The capacitance per unit area of the gate dielectric Cᵢ determines the amount of the charge thus induced. When a negative potential V_{DS} is applied to the drain, the accumulated carriers yield a source-drain current I_{DS} which depends primarily on the density of accumulated carriers and, importantly, their mobility in the source-drain channel. Geometric factors such as the drain and source electrode configuration, size and distance also affect the current. Typically a range of gate and drain voltages are scanned during the study of the device. The source-drain current is described by Equation (1):

$${I}_{DS} = \frac{μ {WC}_{i}}{L} ( ( {V}_{G} - {V}_{0} ) {V}_{DS} - \frac{{V}_{DS}^{2}}{2} ) + {I}_{Ω} \qquad ( 1 )$$

where V₀ is an offset voltage and I_{Ω} is an ohmic current independent of the gate voltage and is due to the finite conductivity of the material. The other parameters are as defined above.

For the electrical measurements the transistor sample is mounted in a sample holder. Microprobe connections are made to the gate, drain and source electrodes using Karl Suss PH100 miniature probe-heads. These are linked to a Hewlett-Packard 4155B parameter analyser. The drain voltage is set to −5 V and the gate voltage is scanned from +20 to −60V and back to +20V in 1 V steps. In accumulation, when |V_{G}|>|V_{DS}| the source-drain current varies linearly with V_{G}. Thus the field effect mobility can be calculated from the gradient (S) of I_{DS} vs. V_{G} given by Equation (2):

$$S = \frac{μ {WC}_{i} {V}_{DS}}{L} \qquad ( 2 )$$

All field effect mobilities quoted below are calculated using this regime (unless stated otherwise). Where the field effect mobility varies with gate voltage, the value is taken as the highest level reached in the regime where |V_{G}|>|V_{DS}| in accumulation mode. The values quoted below are an average taken over several devices (fabricated on the same substrate):

| Example | Average saturated mobility (cm²/Vs) |
|---|---|
| 1 | 0.53 |
| 2 | 0.00003 |
| 3 | 0.05 |
| 4 | 0.005 |
| 5 | 0.002 |

1. Compounds of formula I 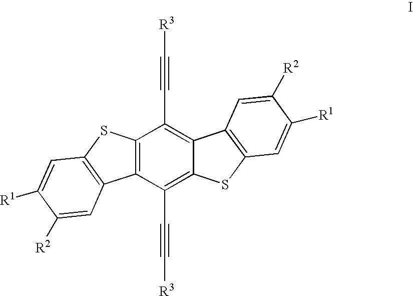
wherein
R¹, R² and R³ are independently of each other halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, optionally substituted silyl groups, or optionally substituted carbyl or hydrocarbyl groups that optionally comprise one or more hetero atoms, neighboured groups R¹ and R² may also form a ring system with each other or with the benzene ring to which they are attached, and R¹ and/or R² may also denote H,
X⁰ is halogen,
R⁰ and R⁰⁰ are independently of each other H or an optionally substituted aliphatic or aromatic hydrocarbyl group having 1 to 20 C atoms.

2. Compounds according to claim 1, characterized in that R³ is a silyl group of the formula SiR′R″R′″, or aryl or heteroaryl group optionally substituted by one or more groups L, wherein
R′, R″, R′″ are identical or different groups selected from H, a C₁-C₄₀-alkyl group, a C₂-C₄₀-alkenyl group, a C₆-C₄₀-aryl group, a C₆-C₄₀-arylalkyl group, a C₁-C₄₀-alkoxy or -oxaalkyl group, or a C₆-C₄₀-arylalkyloxy group, wherein all these groups are optionally substituted by with one or more groups L, or one or more of R′, R″ and R′″ form a cyclic silyl alkyl group together with the Si atom,
L is selected from F, Cl, Br, I, —CN, —NO₂, —NCO, —NCS, —OCN, —SCN, —C(═O)NR⁰R⁰⁰, —C(═O)X⁰, —C(═O)R⁰, —NR⁰R⁰⁰, optionally substituted silyl, aryl or heteroaryl with 4 to 40 C atoms, and straight chain or branched alkyl, alkoxy, oxaalkyl, thioalkyl, alkenyl, alkynyl, alkylcarbonyl, alkoxycarbonyl, alkylcarbonlyoxy or alkoxycarbonyloxy with 1 to 20 C atoms, wherein one or more H atoms are optionally replaced by F or Cl, wherein X⁰, R⁰ and R⁰⁰ are as defined in claim 1.

3. Compounds according to claim 1, characterized in that they are selected from the following subformulae: 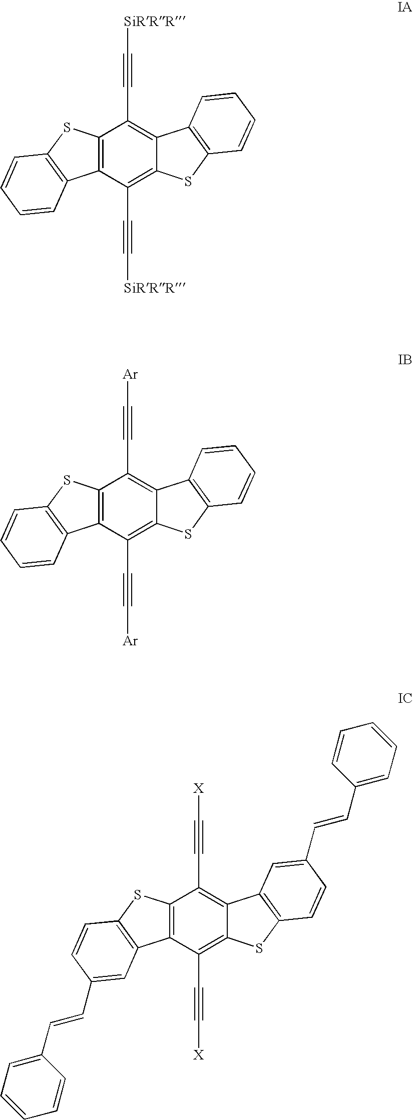 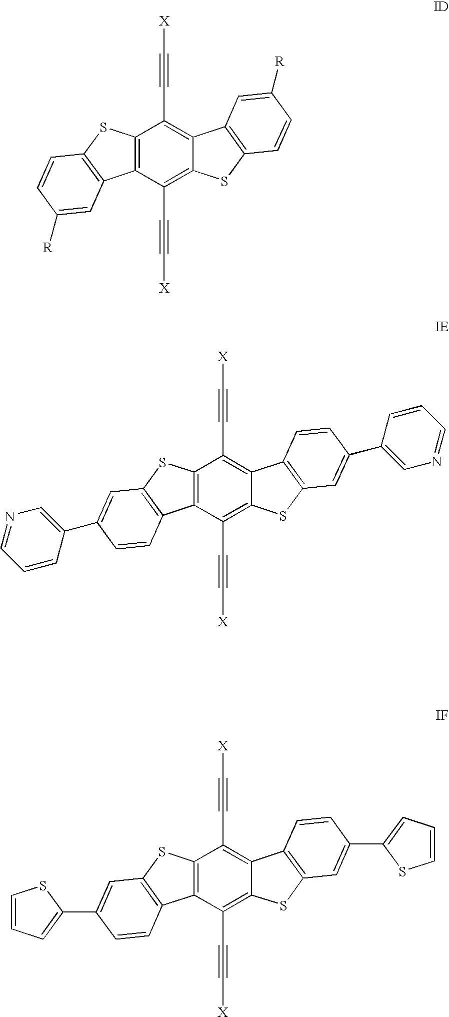
wherein R′, R″ and R′″ are
identical or different groups selected from H, a C₁-C₄₀-alkyl group, a C₂-C₄₀-alkenyl group, a C₆-C₄₀-aryl group, a C₆-C₄₀-arylalkyl group, a C₁-C₄₀-alkoxy or -oxaalkyl group, or a C₆-C₄₀-arylalkyloxy group, wherein all these groups are optionally substituted by with one or more groups L, or one or more of R′, R″ and R′″ form a cyclic silyl alkyl group together with the Si atom,
R has one of the meanings of R² given in claim 1 different from H, X denotes SiR′R″R′″ or Ar, and Ar is in each occurrence independently of one another aryl or heteroaryl group optionally substituted by L
selected from F, Cl, Br, I, —CN, —NO₂, —NCO, —NCS, —OCN, —SCN, —C(═O)NR⁰R⁰⁰, —C(═O)X⁰, —C(═O)R⁰, —NR⁰R⁰⁰, optionally substituted silyl, aryl or heteroaryl with 4 to 40 C atoms, and straight chain or branched alkyl, alkoxy, oxaalkyl, thioalkyl, alkenyl, alkynyl, alkylcarbonyl, alkoxycarbonyl, alkylcarbonlyoxy or alkoxycarbonyloxy with 1 to 20 C atoms, wherein one or more H atoms are optionally replaced by F or Cl, wherein X⁰, R⁰ and R⁰⁰ are as defined in claim 1.

4. Compounds according to claim 3, characterized in that they are selected from the following subformulae: 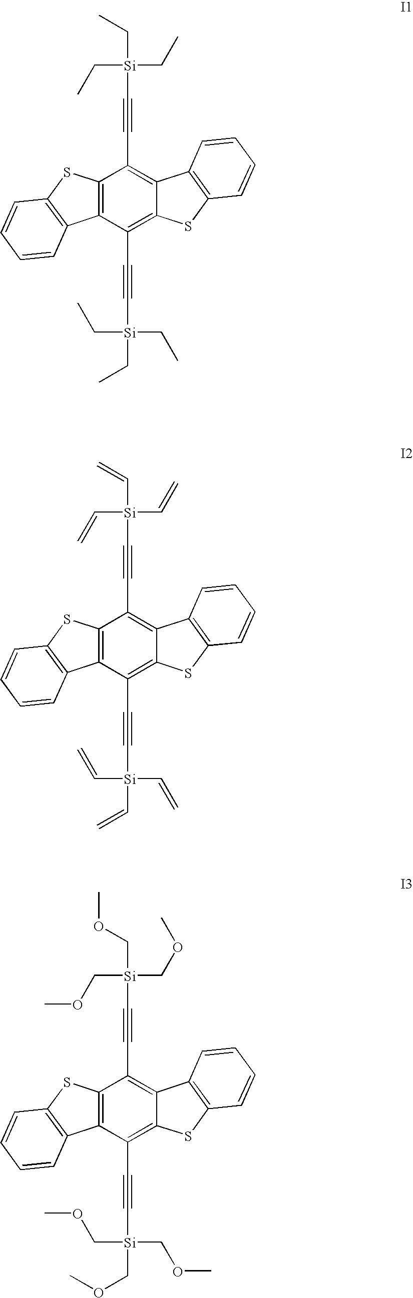 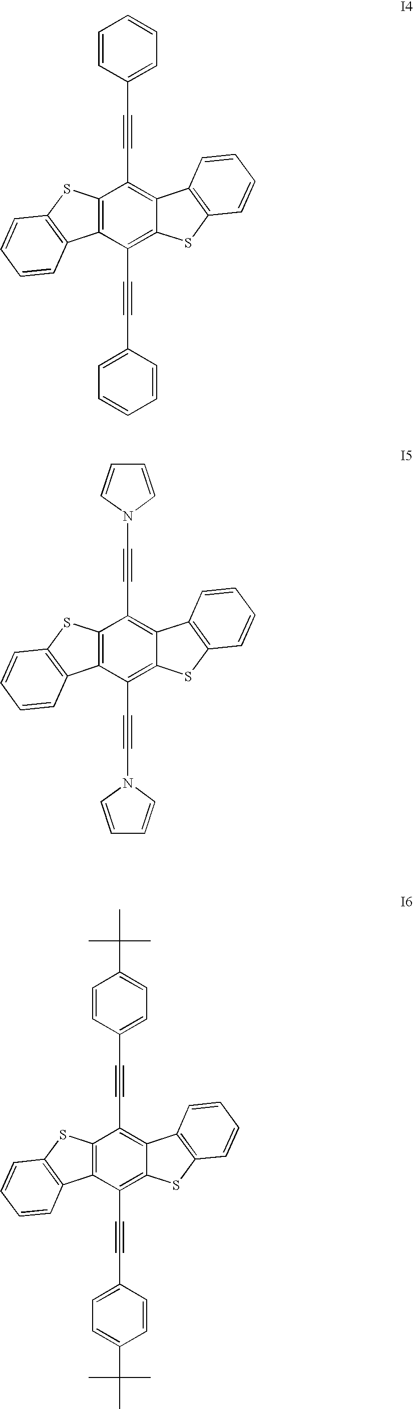 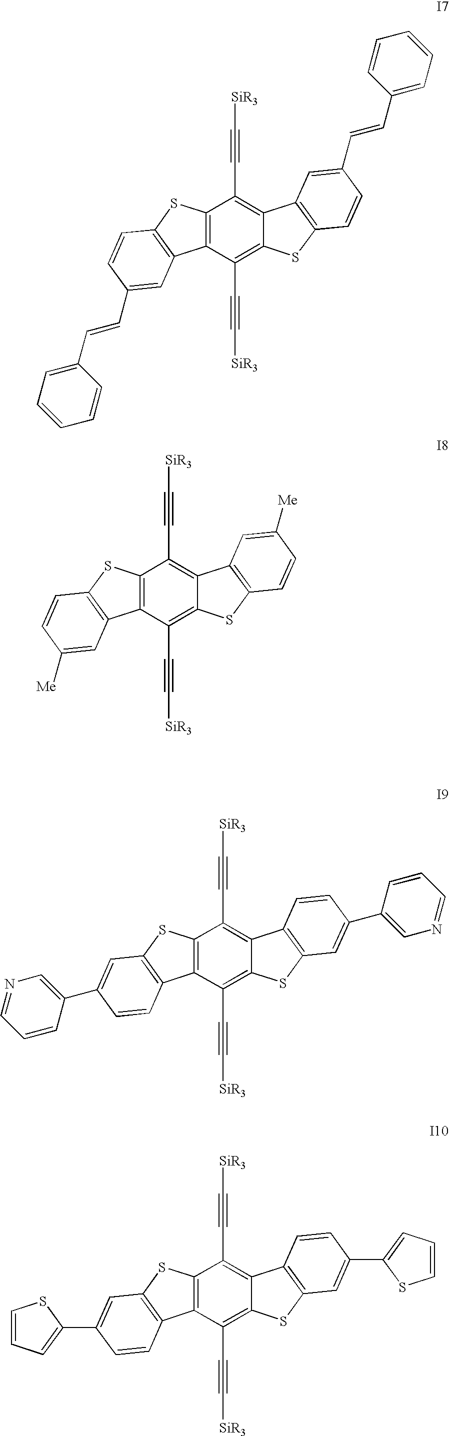 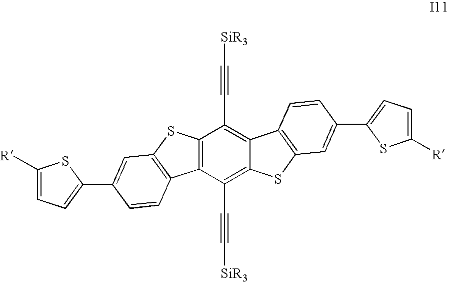
wherein R and R′ are as defined in claim 3.

5. Semiconductor or charge transport material, component or device comprising one or more compounds according to claim 1.

6. Formulation comprising one or more compounds according to claim 1 and one or more organic solvents.

7. Organic semiconducting formulation comprising one or more compounds according to claim 1, one or more organic binders, or precursors thereof, preferably having a permittivity ∈ at 1,000 Hz of 3.3 or less, and optionally one or more solvents.

8. Use of compounds and formulations according to claim 1, as charge transport, semiconducting, electrically conducting, photoconducting or light emitting material in an optical, electrooptical, electronic, electroluminescent or photoluminescent components or devices.

9. Charge transport, semiconducting, electrically conducting, photoconducting or light emitting material or component comprising one or more compounds or formulations according to claim 1.

10. Optical, electrooptical, electronic, electroluminescent or photoluminescent component or device comprising one or more compounds or formulations according to claim 1.

11. Component or device according to claim 10, characterized in that it is selected from electrooptical displays, LCDs, optical films, retarders, compensators, polarisers, beam splitters, reflective films, alignment layers, colour filters, holographic elements, hot stamping foils, coloured images, decorative or security markings, LC pigments, adhesives, non-linear optic (NLO) devices, optical information storage devices, electronic devices, organic semiconductors, organic field effect transistors (OFET), integrated circuits (IC), thin film transistors (TFT), Radio Frequency Identification (RFID) tags, organic light emitting diodes (OLED), organic light emitting transistors (OLET), electroluminescent displays, organic photovoltaic (OPV) devices, organic solar cells (O-SC), organic laser diodes (O-laser), organic integrated circuits (O-IC), lighting devices, sensor devices, electrode materials, photoconductors, photodetectors, electrophotographic recording devices, capacitors, charge injection layers, Schottky diodes, planarising layers, antistatic films, conducting substrates, conducting patterns, photoconductors, electrophotographic devices, organic memory devices, biosensors or biochips.

12. Method of preparing a compound according to claim 1, comprising the following steps:
treatment of an optionally substituted benzo[b]thiophene-3-carboxylic acid dialkylamide or naphtho[2,3-b]thiophene-3-carboxylic acid dialkylamide with at least one equivalent of a strong base at low temperature,
subsequent heating of the resulting organolithium intermediate to promote a first intramolecular condensation reaction between the aryllithium and the carboxylic dialkylamide of another molecule to generate a substituted ketone, and a second intermolecular reaction between the aryllithium and carboxylic acid dialkylamide of the resulting ketone to generate a fused aromatic quinone,
treatment of the resultant fused aromatic quinone with at least two equivalents of an optionally substituted alkynyl lithium or alkynyl magnesium reagent,
nucleophilic addition of the organometallic species to the carbonyl groups, followed by acidic work-up of the resulting diol, optionally in the presence of a reducing agent.
